(19) United States
(12) Patent Application Publication
(10) Pub. No.: US 2015/0042950 A1
(43) Pub. Date: Feb. 12, 2015

(54) OPHTHALMOLOGIC APPARATUS

(71) Applicant: CANON KABUSHIKI KAISHA, Tokyo (JP)

(72) Inventor: Shoichi Yamazaki, Yokohama-shi (JP)

(21) Appl. No.: 14/451,674

(22) Filed: Aug. 5, 2014

(30) Foreign Application Priority Data
Aug. 8, 2013 (JP) 2013-164805

(51) Int. Cl. A61B 3/12; G02B 27/00; A61B 3/10

(52) U.S. Cl. CPC A61B 3/1225; A61B 3/1015; G02B 27/0018
USPC 351/206

(57) ABSTRACT

There is provided a fundus imaging apparatus which suppresses generation of a ghost image, and is capable of high-magnification/high-resolution fundus imaging (AO-SLO), and low-magnification/wide-angle fundus imaging for wide-angle monitoring by a compact optical system having high optical performance. In a fundus imaging apparatus which guides light emitted by a light source to an eye to be inspected, through a scanning unit for two-dimensionally scanning a fundus, and obtains a fundus image based on the light reflected by the eye to be inspected, an optical system between the scanning unit and the eye to be inspected is constituted by a plurality of reflecting surfaces. The first reflecting surface from the eye to be inspected is a rotationally asymmetrical aspherical surface.

### BACKGROUND OF THE INVENTION

1. Field of the Invention

The present invention relates to an ophthalmologic apparatus exemplified by a fundus imaging apparatus and, more particularly, to a fundus imaging apparatus which corrects the aberration of the eyeball optical system of an object and images the small portion of the fundus at high resolution.

2. Description of the Related Art

Recently, an SLO (Scanning Laser Ophthalmoscope) which two-dimensionally irradiates a fundus with a laser beam and receives the reflected light to image the fundus, and an imaging apparatus using the interference of low-coherence light have been developed as ophthalmologic apparatuses serving as ophthalmologic imaging apparatuses.

The imaging apparatus using the interference of low-coherence light is called an OCT (Optical Coherence Tomography: optical coherence tomography apparatus or optical coherence tomography method), and is used to especially obtain the tomogram of a fundus or its vicinity. Various kinds of OCTs have been developed, including a TD-OCT (Time Domain OCT: time domain method) and an SD-OCT (Spectral Domain OCT: spectral domain method). Recently, the resolution of particularly an ophthalmologic imaging apparatus like this has been increased by increasing the NA of an irradiation laser or the like.

However, the fundus must be imaged through the optical tissues of an eye such as the cornea and crystalline lens. As the resolution increases, the aberrations of the cornea and crystalline lens exert a large influence on the image quality of an acquired image. Accordingly, an AO-SLO and AO-OCT incorporating an adaptive optics (AO) function of measuring and correcting the aberration of an eye have been studied.

The AO-SLO and AO-OCT generally measure the wavefront aberration of an eye by using a Shack-Hartmann wavefront sensor system. The Shack-Hartmann wavefront sensor system measures the wavefront aberration by irradiating an eye with measurement light, and receiving the reflected light by a CCD camera through a microlens array. A wavefront correction device such as a variable shape mirror or spatial phase modulator (e.g., liquid crystal) is driven to correct the measured wavefront aberration, and the fundus is imaged upon canceling the aberration of the eye. Consequently, the AO-SLO and AO-OCT can perform high-resolution fundus imaging.

These optical systems are configured by arranging a scanning unit (X/Y scanner), wavefront correction device, and wavefront sensor sequentially from the eyeball side at a position conjugate to the pupil of an eyeball, and image the pupil through these building components. The optical systems of the AO-SLO and AO-OCT generally use a spherical lens/aspherical lens and a spherical reflecting mirror/aspherical reflecting mirror. When a lens is used as an element of the optical system, the surface reflection of each lens surface is 4%. This reflected light is 20 times larger than weak fundus reflected light used in imaging at a reflectance of about 0.2%, and is considerably strong light. For this reason, the lens surface reflection generates an undesirable ghost image. If the optical system is constituted by a reflecting mirror, such a ghost image is not generated, but the reflecting mirror needs to be tilted eccentrically. The optical system thus becomes an eccentric optical system, and an eccentric aberration is generated in addition to general aberrations (Seidel's five aberrations), failing in obtaining a satisfactory image. The former optical system weakens a ghost image by coating the lens surface to decrease the surface reflection, but the ghost image is not completely canceled. The latter optical system cannot correct a generated eccentric aberration in principle by a general spherical surface or aspherical surface (rotational symmetry about the optical axis of the surface). Therefore, the latter optical system generally obtains a satisfactory image by weakening the optical refractive power of the reflecting mirror surface, decreasing the tilt eccentric amount, and suppressing the eccentric aberration. However, this upsizes the optical system.

An invention disclosed in Japanese Patent Application Laid-Open No. 2010-259669 employs an eccentric optical system constituted by a reflecting mirror, and adopts a free-form surface having a special surface shape on the reflecting surface of the reflecting mirror. A general spherical or aspherical surface is a rotationally symmetrical surface whose surface shape remains unchanged even upon rotation about the optical axis. However, the free-form surface is a rotationally asymmetrical surface because the surface shape changes upon rotation about the optical axis of the surface. FIG. 4 shows an example of the surface shape of a rotationally asymmetrical three-dimensional aspherical surface (free-form surface). Such a free-form surface can correct the eccentric aberration which has not been corrected by a general spherical surface or aspherical surface.

In the invention disclosed in Japanese Patent Application Laid-Open No. 2010-259669, a free-form reflecting surface is employed between a scanning unit (scanner) and a wavefront correction device which increases the diameter of a laser beam on the optical path. This arrangement effectively suppresses the eccentric aberration and achieves both downsizing and high optical performance.

For the AO-SLO and AO-OCT, there is also proposed, for example, an arrangement disclosed in Japanese Patent Application Laid-Open No. 2010-259543 in which a wide-angle monitoring function of imaging an entire fundus by a fundus imaging optical system having a low magnification and a wide angle of view is arranged in addition to a function of imaging the small portion of the fundus by adaptive optics at a high magnification and a high resolution.

In a present AO-SLO, the need for wide-angle monitoring as exemplified in Japanese Patent Application Laid-Open No. 2010-259543 is growing in addition to the high-magnification/high-resolution imaging unit. In the arrangement disclosed in Japanese Patent Application Laid-Open No. 2010-259669, aberrations including a high-order eccentric aberration are satisfactorily corrected by employing the free-form surface on a plane between the scanning unit (scanner) and the wavefront correction device which enlarges a beam on the optical path. However, the imaging angle on the eyeball side is as small as ±3°, so it is difficult to achieve both the high-magnification/high-resolution imaging function and the function of the wide-angle imaging optical system requiring a minimum of ±10° or more.

According to the present invention, the deflection angle mode of a scanning unit (scanner) in an eccentric mirror optical system includes two modes: a high-magnification/high-resolution imaging mode using a small deflection angle, and a low-magnification/wide-angle imaging mode using a large deflection angle. As the eccentric optical system, a common optical system is provided for both high-resolution imaging and wide-angle imaging. In the invention shown in the Japanese Patent Application Laid-Open No. 2010-259669, the imaging angle of view on the eyeball side is as small as ±3° because of the high-magnification/high-resolution optical system (AO-SLO). For this reason, the free-form surface is employed on a plane between the scanning unit (scanner) and the wavefront correction device which enlarges a beam on the optical path. Accordingly, aberrations including even a high-order eccentric aberration can be satisfactorily corrected, and downsizing can also be implemented. However, the eccentric optical system according to the present invention needs to have even the function of the wide-angle imaging optical system. For wide-angle imaging, the imaging angle of view on the eyeball side needs to be a minimum of ±10° or more. As the imaging angle of view increases, the generation amount of eccentric aberration greatly increases. It is therefore necessary to suppress the eccentric aberration at each angle of view between the pupil of the eyeball and the scanner which generates a beam at each angle of view.

### SUMMARY OF THE INVENTION

The present invention has been made in consideration of the above situation, and provides an ophthalmologic apparatus which provides a high-magnification/high-resolution fundus imaging optical system (AO-SLO) and low-magnification/wide-angle fundus imaging optical system by one optical system as a reflecting mirror optical system free from generation of a ghost image, and thus can implement downsizing and high performance.

In a fundus imaging apparatus which guides light emitted by a light source to an eye to be inspected, through a scanning unit for two-dimensionally scanning a fundus, and obtains a fundus image based on the light reflected by the eye to be inspected, an optical system between the scanning unit and the eye to be inspected is constituted by a plurality of reflecting surfaces, and the first reflecting surface from the eye to be inspected is a rotationally asymmetrical aspherical surface.

In the optical system constituted by the plurality of reflecting surfaces between the scanning unit and the eye to be inspected, a reflecting surface having a strongest optical refractive power on an eccentric section (meridional section) is a rotationally asymmetrical aspherical surface.

A wavefront correction device is interposed between the scanning unit and the light source. Wavefront data which worsens depending on the manufacturing error of the rotationally asymmetrical aspherical surface exists and is corrected by the wavefront correction device.

In the optical system constituted by the plurality of reflecting surfaces between the scanning unit and the eye to be inspected, a light wavelength division unit is interposed between the scanning unit and two or more rotationally asymmetrical aspherical surfaces from the eye to be inspected.

The present invention can provide a compact fundus imaging apparatus having high optical performance while suppressing generation of a ghost image. In addition, one optical system can implement the function of a high-magnification/high-resolution fundus imaging optical system (AO-SLO) and the function of a low-magnification/wide-angle fundus imaging optical system for wide-angle monitoring.

Further features of the present invention will become apparent from the following description of exemplary embodiments with reference to the attached drawings.

### BRIEF DESCRIPTION OF THE DRAWINGS

FIG. 1 is a view showing an arrangement according to the first embodiment of the present invention.

FIG. 2 is a view showing an optical path according to the numerical embodiment of the first embodiment of the present invention.

FIG. 3 is a view showing an arrangement according to the second embodiment of the present invention.

FIG. 4 is a view exemplifying the surface shape of a free-form surface.

### DESCRIPTION OF THE EMBODIMENTS

Preferred embodiments of the present invention will now be described in detail in accordance with the accompanying drawings.

The present invention employs a free-form surface as the first reflecting surface closest to an eye to be inspected, between a scanning unit and the eye to be inspected, that is, a reflecting surface which first reflects light reflected by the eye to be inspected, among a plurality of reflecting surfaces arranged in an ophthalmologic apparatus. As described above, an eccentric aberration is generated in a reflecting mirror system, and the generation amount of eccentric aberration becomes larger as the angle of view becomes larger. To correct the eccentric aberration at a large angle of view, the free-form surface needs to be arranged on the eyeball side with respect to the scanning unit for generating a beam at each angle of view. Since eccentric aberration correction amounts at respective angles of view are different, areas corresponding to beams at respective angles of view in the effective area of the free-form surface are desirably separated without overlapping each other. To correct eccentric aberrations including a high-order one on the free-form surface, the diameter of a beam at each angle of view is desirably large. From this viewpoint, on the reflecting surface closest to the eyeball, the areas of beams at respective angles of view are spaced apart from each other, and the diameter of a beam at each angle of view becomes largest between the scanning unit and an eye to be inspected (see s2 in an embodiment of FIG. 2). By placing the free-form surface at this position, an eccentric aberration at each angle of view is most satisfactorily corrected.

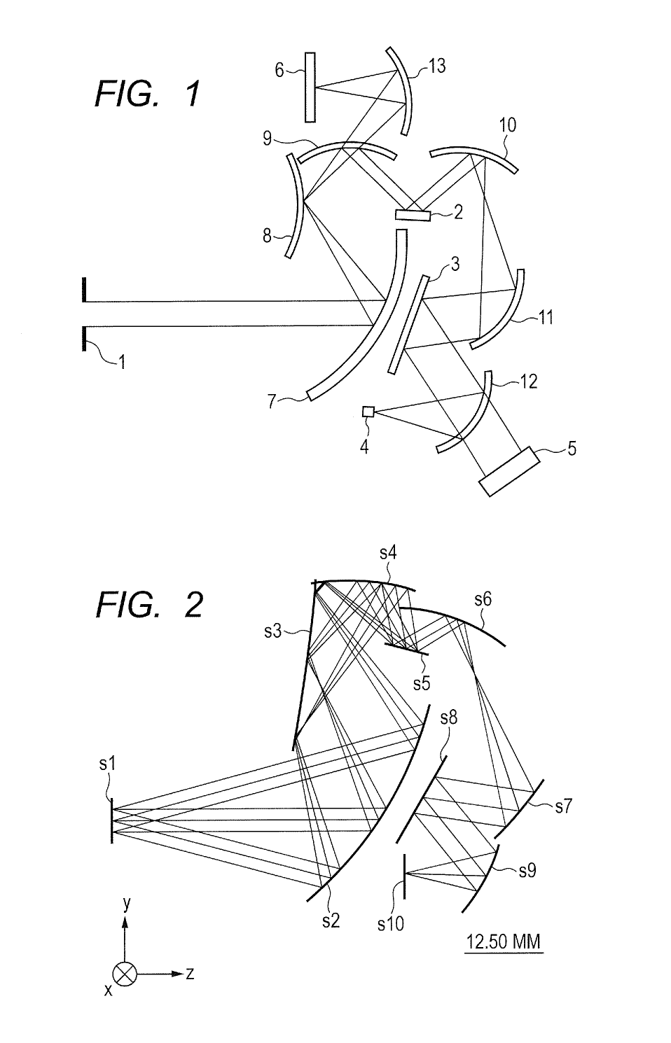

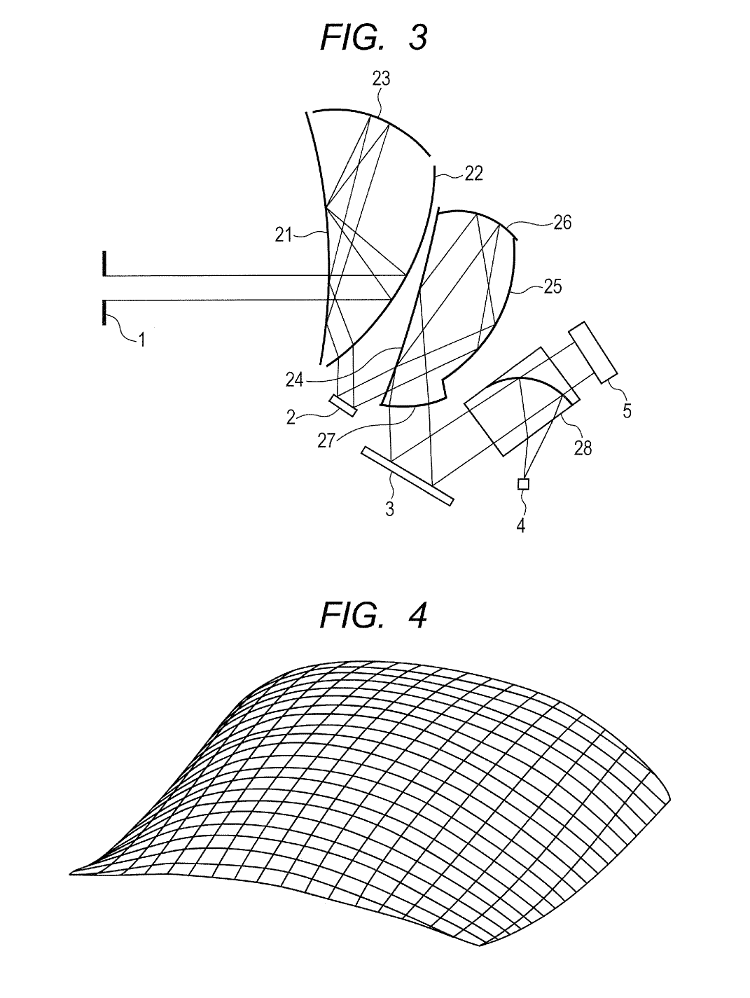

The present invention employs a free-form surface as a surface having a strongest optical refractive power on an eccentric section (meridional section) between the scanning unit and an eye to be inspected (s2 in FIG. 2). FIGS. 1 and 2 show a reflecting optical system according to the present invention. The reflecting optical system is tilted eccentrically on the paper surface, that is, meridional section, and is not eccentric on a section perpendicular to the paper surface=a sagittal section. Therefore, the eccentric aberration is generated on only the meridional section. By increasing the optical refractive power of a surface, the optical system can be downsized. However, as the optical refractive power of a surface becomes stronger, the generation amount of eccentric aberration also becomes larger. Considering this, a free-form surface is adopted as a surface having a strongest optical refractive power on the eccentric section (meridional section). More specifically, the second free-form surface is arranged as a reflecting surface which is arranged on the optical path between the scanning unit and an eye to be inspected (s2, s3, and s4 in FIG. 2), and has a strongest optical refractive power on the eccentric section among a plurality of reflecting surfaces. A surface having the smallest absolute value of a radius Ry of curvature of an actual meridional section (on an eccentric section) among the reflecting surfaces s2, s3, and s4 in Table 1 to be described later is a surface having a strongest refractive power and is s2. By suppressing an eccentric aberration generated on this surface, the optical system can be downsized.

Since the free-form surface is a rotationally asymmetrical three-dimensional aspherical surface, it is difficult to manufacture a surface shape complying with a design value, unlike general rotationally symmetrical spherical and aspherical surfaces. The manufacturing error inevitably becomes large. It is therefore preferable to register the manufacturing error in advance, and correct an aberration or the like arising from the manufacturing error by an arranged wavefront correction device. More specifically, according to the present invention, the wavefront correction device which corrects the wavefront of reflected light or an aberration is inserted in an optical path between the scanning unit and a light source. In addition, a storage unit for storing the optical characteristics of the above-mentioned free-form surface and second free-form surface is arranged. The storage unit is arranged as a component of a control device which controls alignment of an optical system with respect to an eye to be inspected, ON/OFF of the light source and the like, scanning of the scanning unit, and the like. As described above, the wavefront correction device corrects the wavefront of reflected light based on the optical characteristics of the free-form surface that are stored in the storage unit. This makes it possible to use even the free-form surface having a large manufacturing error as a reflecting surface, and greatly reduce the cost.

An AO-SLO apparatus needs to incorporate even a fixation lamp display optical system (to be described later), in addition to the above-mentioned high-magnification/high-resolution optical system and low-magnification/wide-angle fundus imaging optical system. For this purpose, an optical path extending to the fixation lamp display optical system needs to be divided somewhere from the above-described aberration correction-related optical path. At this time, to reduce the light amount loss, the fixation lamp display optical system preferably uses a different wavelength to divide the optical path based on the wavelength difference.

The fixation lamp display optical system must be capable of fixation display at a widest angle of view (angle of view in low-magnification/wide-angle fundus imaging). As a display device, a low-cost two-dimensional display panel (e.g., EL or liquid crystal) is used. The aforementioned wavelength division unit is arranged on the eyeball side with respect to the scanning unit for generating a beam at each angle of view. At this position, the optical path of the fixation lamp display optical system needs to be separated. At this time, the optical system is or is not shared between the fixation lamp display optical system, and the high-magnification/high resolution optical system and low-magnification/wide-angle optical system. When the optical system is not shared, the wavelength division unit is arranged on the most eyeball side to divide the optical path. In this case, the high-magnification/high-resolution optical system and low-magnification/wide-angle optical system are spaced apart from the eyeball to interpose the wavelength division unit between them. As a result, the high-magnification/high-resolution optical system and low-magnification/wide-angle optical system undesirably become very large. To prevent this, part of the optical system including a free-form surface between the eyeball and the scanning unit is shared, and the wavelength division unit is arranged behind it (9 in FIG. 1 and s4 in FIG. 2).

The free-form surface can correct an eccentric aberration, but the eccentric aberration inevitably remains by one free-form surface. If the free-form surface has a strong refractive power, the eccentric aberration remains still more. In the present invention, therefore, another free-form reflecting surface is arranged (8 in FIG. 1 and s3 in FIG. 2), in addition to the first reflecting surface (free-form surface: 7 in FIG. 1 and s2 in FIG. 2) from the eyeball side. Such a surface shape of the free-form surface as to cancel the remaining eccentric aberration is given to greatly reduce the eccentric aberration. If the wavelength division unit is arranged behind this reflecting surface, downsizing becomes possible with a simple arrangement without requiring a free-form surface and many reflecting surfaces in the fixation lamp display optical system on the divided optical path (13 in FIG. 1). That is, the light wavelength division unit for guiding an optical path from the fixation lamp display optical system to an eye to be inspected is preferably interposed between the scanning unit, and at least two free-form reflecting surfaces from an eye to be inspected, among a plurality of reflecting surfaces interposed between the scanning unit and the eye to be inspected.

### First Embodiment

The first embodiment of the present invention will now be described with reference to the accompanying drawings. The embodiment shown in FIG. 1 will describe an ophthalmologic apparatus which obtains an image based on light which has been emitted by a light source and reflected by an eye to be inspected. The ophthalmologic apparatus includes a plurality of reflecting surfaces which reflect the light or the reflected light, and a scanning unit which scans the light on a fundus. In FIG. 1, a scanning unit (XY scanner: to be referred to as a scanner hereinafter) 2, and a wavefront correction device (LCOS) 3 form the image of a pupil at a position conjugate to a pupil 1 of the eye to be inspected. Note that a Shack-Hartmann (SH) sensor 5 desirably forms an image at a position conjugate to the pupil 1, but may not be conjugate. In the embodiment, the SH sensor 5 is not conjugate, and a sensor output is corrected. A fixation lamp display panel 6 displays an indicator, mark, or the like to prompt the object to, for example, look up or left. By changing the indicator position, a different location of the fundus is imaged.

Light emitted by an 840-nm SLD light source 4 is reflected by a free-form half mirror 12, becomes almost parallel light, and enters the wavefront correction device 3. The wavefront correction device 3 is a LCOS liquid crystal element (optical effective diameter of φ6 to φ8 mm), but a deformable mirror may be used. The light reflected by the wavefront correction device 3 is converged by a concave curved mirror 11, returned again to parallel light by a concave curved mirror 10, enters the scanner 2, and is scanned in the x and y directions, generating a beam at each angle of view. In the present invention, one biaxial (x and y) scanner is used as the scanner 2. Alternatively, two uniaxial scanners may be used and arranged close in the vertical direction. Note that the scanner 2 can switch between deflection angles in the two modes. The scanner 2 deflects light at ±4° in high-magnification/high-resolution fundus imaging (AO-SLO) (an imaging angle of view of ±3°), and at ±20° in low-magnification/wide-angle fundus imaging (an imaging angle of view of ±150) (the pupil has φ4 mm, and the optical effective diameter of the scanner 2 is φ3 mm). It is also possible to set three or more modes and deflect light by the scanner 2 at ±13° in imaging at an angle of view of ±10°. As for the deflection angle when scanning light on an eye to be inspected, the scanner 2 serving as the scanning unit preferably at least switches between the first deflection angle and the second deflection angle larger than the first deflection angle. The scanner 2 generates beams of parallel light at respective angles of view. A light wavelength division free-form mirror (light wavelength division mirror) 9 reflects and converges these beams. A dichroic film is formed on the light wavelength division mirror 9, reflects light around 840 nm, and transmits light having the wavelength of visible light used for fixation lamp display. The light reflected and converged by the light wavelength division mirror 9 is reflected by a free-form mirror 8, and further changed into parallel light by a free-form mirror 7 arranged as a reflecting surface having a strongest optical refractive power among reflecting surfaces between the scanner 2 and the pupil 1. The parallel light enters the pupil (φ4 mm) of the eye to be inspected and is converged to the fundus. Weak return light (0.2%) traveling from the fundus returns completely reversely, and reaches the light source 4. A fiber coupler (not shown) is arranged behind the light source 4, and emission of light from the light source 4 and reception of light can be performed. The received return light is guided to a sensor (not shown) to image every point. In other words, the light reflected by the eye to be inspected is received by a light receiving unit arranged together with the light source. Of the light returning from the fundus, light having passed through the free-form half mirror 12 is guided to the SH sensor 5 and used to calculate the wavefront aberration of the eye to be inspected. That is, the SH sensor 5 serving as a wavefront measurement unit receives part of reflected light through, e.g., the half mirror (light splitting unit) which is arranged in front of the light receiving unit (not shown) and formed from a free-form surface.

Wavefront data to cancel the wavefront aberration is transferred to the wavefront correction device 3 to perform wavefront correction and execute high-resolution imaging. In the fixation lamp display optical system, light traveling from the display panel (two-dimensional light emitting surface: visible light wavelength) 6 is converged by a concave curved mirror 13, and passes through the light wavelength division mirror 9. After that, the light is changed into parallel light by the free-form mirrors 8 and 7, similarly to 840-nm light, and is converged to the fundus, displaying the indicator.

### Numerical Embodiment of First Embodiment

A numerical embodiment designed with specifications according to the above-described first embodiment is shown in Table 1 and FIG. 2.

s1: the diameter of the pupil 1 is φ4 mm, the imaging angle of view: ±150, s5: the optical effective diameter of the scanner 2 is φ3 mm, s8: the optical effective diameter of the wavefront correction device 3 is φ6 to φ8 mm, s10: the light source/light receiving sensor 4 (resolution of 5 μm)

Table 1 will be explained. s1 to s10 represent the numbers of respective surfaces. The xyz coordinate system is shown in FIG. 2. An eccentric section (section along the paper surface in FIG. 2) is defined as a meridional section, and a section perpendicular to the meridional section is defined as a sagittal section. The radius ry of curvature of the meridional section, the radius rx of curvature of the sagittal section, the surface interval d (distance parallel to the surface vertex coordinate system of the first surface), the eccentric amount (the parallel eccentric amount of the surface vertex of each surface with respect to the surface vertex coordinate system of the first surface on the meridional section is represented by shift, and the tilt eccentric amount (degree) is represented by tilt), and the refractive index n are shown in (general-paraxial axis) of Table 1. FFS stands for a free-form surface (rotationally asymmetrical surface). Further, a surface to which “M” is added on the left end in the table is a reflecting surface, and the refractive index n has an opposite sign.

The definitional equation of FFS (Free-Form Surface) is as follows. The following equation is a definitional equation in the surface vertex coordinate system of each surface.

FFS:

z=(1/R)*(x²+y²)/(1+(1−(1+c1)*(1/R)²*(x²+y²))^{(1/2)})+c5*(x²−y²)+c6*(−1+2*x²+2*y²)+c10*(−2*y+3*x²*y+3*y³)+c11*(3*x²*y−y³)+c12*(x⁴−6*x²*y²+y⁴)+c13*(−3*x²+4*x⁴+3*y²−4*y⁴)+c14*(1−6*x²+6*x⁴−6*y²+12*x²*y²+6*y⁴)+c20*(3*y−12*x²*y+10*x⁴*y−12*y³+20*x²*y³+10*y⁵)+c21*(−12*x²*y+15*x⁴*y+4*y³+10*x²*y³−5*y⁵)+c22*(5*x⁴*y−10*x²*y³+y⁵)+c23*(x⁶−15*x⁴*y²+15*x²*y⁴−y⁶)+c24*(−5*x⁴+6*x⁶+30*x²*y²−30*x⁴*y²−5*y−30*x²*y⁴+6*y⁶)+c25*(6*x²−20*x⁴+15*x⁶−6*y²+15*x⁴*y²+20*y⁴−15*x²*y−15*y⁶)+c26*(−1+12*x²−30*x⁴+20*x⁶+12*y²−60*x²*y²+60*x⁴*y²−30*y⁴+60*x²*y⁴+20*y⁶)+ . . . (1)

In the above definitional equation, cx, cy, c1, c5, . . . are free-form surface coefficients. Since paraxial coefficients (e.g., c1, c5, and c6) exist among the free-form surface coefficients for this free-form surface, the value of the radius ry of curvature of the meridional section and the value of the radius rx of curvature of the sagittal section in (general-paraxial axis) do not express a radius Ry of curvature of an actual meridional section and a radius Rx of curvature of an actual sagittal section at the surface vertex. The radius Ry of curvature of the actual meridional section and the radius Rx of curvature of the actual sagittal section at the surface vertex have different values. Therefore, the radius Ry of curvature of the actual meridional section and the radius Rx of curvature of the actual sagittal section at the surface vertex (x, y)=(0, 0) in the surface vertex coordinate system are calculated and shown. As detailed calculation of Ry and Rx, the radius Ry of curvature on the meridional section is calculated at three points: one point of data at the surface vertex (x, y, z)=(0, 0, 0), and two points of coordinate data (0, ±Δy, z) upon a small displacement ±Δy from the surface vertex in the y direction. Similarly, the radius Rx of curvature on the sagittal section is calculated upon a small displacement ±Δx on the sagittal section in each surface vertex coordinate system.

TABLE 1
(Numerical Embodiment of First-Embodiment)
(general - paraxial axis)

| | n | ry | rx | d | shift | tilt | n |
|---|---|---|---|---|---|---|---|
| | s1 | 0.00000 | 0.00000 | 40.000 | 0.000 | 0.000 | 1.000 |
| FFS-M | s2 | −58.75281 | −58.75281 | −10.000 | −8.435 | −43.000 | −1.000 |
| FFS-M | s3 | −525.66514 | −525.66514 | −5.000 | 3.000 | −10.000 | −1.000 |
| FFS-M | s4 | −238.53780 | −238.53780 | 25.000 | 40.000 | 80.000 | −1.000 |
| M | s5 | 0.00000 | 0.00000 | 10.000 | 30.000 | 75.000 | −1.000 |
| M | s6 | −30.00000 | −30.00000 | 10.000 | 35.000 | 70.000 | −1.000 |
| M | s7 | −60.00000 | −60.00000 | −18.000 | 0.000 | −42.000 | −1.000 |
| M | s8 | 0.00000 | 0.00000 | 10.000 | 0.000 | −30.000 | −1.000 |
| FFS-M | s9 | −28.04328 | −28.04328 | −11.000 | −15.000 | −38.000 | −1.000 |
| | s10 | 0.00000 | 0.00000 | 0.000 | −10.000 | 0.000 | 1.000 |

| | | | | | |
|---|---|---|---|---|---|
| FFS | s2 | c1 = −1.0084e−001 | c5 = −1.0697e−003 | c6 = −2.4351e−004 | c10 = 4.5857e−008 |
| | | c11 = 8.5030e−006 | c12 = −1.3060e−007 | c13 = −5.3118e−008 | c14 = 9.9555e−009 |
| | | c20 = 3.2814e−010 | c21 = −5.1271e−010 | c22 = 1.9018e−010 | c23 = 1.2849e−011 |
| | | c24 = 9.4531e−012 | c25 = −7.2342e−012 | c26 = 3.4030e−012 | |
| FFS | s3 | c1 = −9.5064e+001 | c5 = 1.5215e−004 | c6 = 4.7311e−004 | c10 = 1.9422e−006 |
| | | c11 = 1.2707e−005 | c12 = −1.5286e−007 | c13 = 7.4555e−009 | c14 = 9.8657e−009 |
| | | c20 = −2.3703e−011 | c21 = 2.5590e−010 | c22 = −1.2138e−009 | c23 = 1.1628e−011 |
| | | c24 = −5.2420e−012 | c25 = 4.6287e−013 | c26 = −1.4156e−013 | |
| FFS | s4 | c1 = 5.0001e+001 | c5 = −4.9927e−004 | c6 = −1.0986e−003 | c10 = 1.9390e−005 |
| | | c11 = 1.9736e−005 | c12 = 5.8248e−007 | c13 = 1.4892e−007 | c14 = −2.2299e−007 |
| | | c20 = 2.4445e−009 | c21 = −4.0802e−010 | c22 = −1.5534e−008 | c23 = −4.2072e−010 |
| | | c24 = 3.1903e−011 | c25 = 2.7024e−011 | c26 = −2.9357e−011 | |
| FFS | s9 | c1 = −7.6484e−002 | c5 = −4.6905e−003 | c6 = −9.7853e−004 | c10 = −6.0454e−005 |
| | | c11 = 1.4259e−005 | c12 = 4.0834e−006 | c13 = −1.1012e−006 | c14 = 4.7122e−007 |
| | | c20 = 6.0957e−008 | c21 = 3.1008e−008 | c22 = −1.9767e−007 | c23 = −3.3413e−009 |
| | | c24 = 1.7602e−009 | c25 = 2.2413e−011 | c26 = −1.2000e−009 | |

| n | point (y, x) | Ry | Rx |
|---|---|---|---|
| s2 | (0.000, 0.000) | −63.069 | −49.668 |
| s3 | (0.000, 0.000) | −3181.807 | 3398.697 |
| s4 | (0.000, 0.000) | −131.851 | −104.349 |
| s5 | (0.000, 0.000) | 0.000 | 0.000 |
| s6 | (0.000, 0.000) | −30.000 | −30.000 |
| s7 | (0.000, 0.000) | −60.000 | −60.000 |
| s8 | (0.000, 0.000) | 0.000 | 0.000 |
| s9 | (0.000, 0.000) | −33.108 | −20.428 |

### Second Embodiment

The second embodiment is the same as the first embodiment in specifications such as the light source wavelength, a pupil 1, a scanner 2, a wavefront correction device 3, a light source 4, a light receiving sensor (not shown), and an SH sensor 5. Reflecting surfaces are constituted by free-form mirrors or curved mirrors in the first embodiment, but are constituted by three prisms in the second embodiment. Light emitted by the light source 4 is reflected by and emerges from a prism 28 joined by a free-form surface half mirror. The light is then reflected by the wavefront correction device 3, and enters the transmissive refraction free-form surface of a prism 27 constituted by four free-form surfaces. Total reflection free-form surfaces 24 and 25 have large incident angles exceeding the total reflection angle, and thus totally reflect the measurement light without reflective coating. When emerging from the prism 27, the light is transmitted and refracted to become parallel light and emerge from the prism 27 because the angle of incidence on the total reflection free-form surface 24 is smaller than the total reflection angle. The parallel light is reflected by the scanner 2 to generate a beam at each angle of view. The beam enters a transmissive refraction portion, which does not have a mirror below a free-form mirror, of a prism 22 constituted by three free-form surfaces. Similarly to the total reflection free-form surfaces 24 and 25, a total reflection free-form surface 21 does not undergo reflective coating, and totally reflects light. Also, when the parallel light emerges, the light is transmitted and refracted to become parallel light and emerge because the angle of incidence on the total reflection free-form surface 21 is smaller than the total reflection angle. The light is guided to an eye to be inspected. The light similarly returns from the fundus, and is guided to the light receiving sensor (4) and the SH sensor 5. A free-form mirror 23 is a light wavelength division mirror (dichroic mirror). As in the first embodiment, a fixation lamp display system having a visible light wavelength may be arranged at a position at which light passes through the free-form mirror 23.

### Other Embodiments

The present invention is also implemented by executing the following processing. More specifically, software (program) for implementing the functions of the above-described embodiments is supplied to a system or apparatus via a network or various storage media, and the program is read out and executed by the computer (e.g., CPU or MPU) of the system or apparatus.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-164805, filed Aug. 8, 2013, which is hereby incorporated by reference herein in its entirety.

What is claimed is:

1. An ophthalmologic apparatus which obtains an image based on light that has been emitted by a light source and reflected by an eye to be inspected, comprising:
a plurality of reflecting surfaces each configured to reflect the light; and
a scanning unit configured to scan the light on a fundus of the eye to be inspected,
wherein, among the plurality of reflecting surfaces, a reflecting surface which first reflects the light reflected by the eye to be inspected is a free-form surface.

2. An apparatus according to claim 1, wherein, among the plurality of reflecting surfaces, a reflecting surface which is inserted in an optical path between the scanning unit and the eye to be inspected and has a strongest optical refractive power on an eccentric section is a second free-form surface.

3. An apparatus according to claim 1, further comprising:
a wavefront correction device inserted in an optical path between the scanning unit and the light source and configured to perform wavefront correction of the reflected light; and
a storage unit configured to store optical characteristics of the free-form surface and second free-form surface,
wherein the wavefront correction device performs wavefront correction of the reflected light based on the stored optical characteristics.

4. An apparatus according to claim 1, further comprising a light wavelength division unit interposed between the scanning unit and the reflecting surfaces serving as at least two free-form surfaces from the eye to be inspected among the plurality of reflecting surfaces interposed between the scanning unit and the eye to be inspected, and configured to separate light of a predetermined wavelength.

5. An apparatus according to claim 1, further comprising:
a light receiving unit arranged together with the light source and configured to receive the reflected light; and
a wavefront aberration measurement unit configured to receive part of the reflected light through a light splitting unit arranged in front of the light receiving unit and formed from a free-form surface.

6. An apparatus according to claim 1, wherein as for a deflection angle when scanning the light on the eye to be inspected, the scanning unit can switch between a first deflection angle and a second deflection angle larger than the first deflection angle.

7. An apparatus according to claim 1, wherein the plurality of reflecting surfaces include a reflecting surface constituted by a reflecting mirror.

8. An apparatus according to claim 1, wherein the plurality of reflecting surfaces include a reflecting surface formed on a prism.
